SEQUENCE LISTING (1 sequence)

SEQ ID NO: 1 - DNA, 10 bp, Artificial Sequence
  Barcode sequence
acgtagtacg

(19) United States
(12) Patent Application Publication
(10) Pub. No.: US 2024/0257914 A1
(43) Pub. Date: Aug. 1, 2024

(54) METHOD AND SYSTEM FOR 3D RECONSTRUCTION OF TISSUE GENE EXPRESSION DATA

(71) Applicant: MAX-DELBRÜCK-CENTRUM FÜR MOLEKULARE MEDIZIN IN DER HELMHOLTZ-GEMEINSCHAFT, BERLIN (DE)

(72) Inventors: JONATHAN ALLES, MAINZ (DE); NIKOLAOS KARAISKOS, BERLIN (DE); NIKOLAUS RAJEWSKY, BERLIN (DE); GIUSEPPE MACINO, TORRITA TIBERINA (IT); ANDREW WOEHLER, BERLIN (DE); ENES SENEL, BERLIN (DE); STEFANO ABBIATI, BERGAMO (IT); SALAH AYOUB, BERLIN (DE); SEBASTIAN EHRIG, POTSDAM (DE); STEPHAN PREIBISCH, ASHBURN, VA (US)

(21) Appl. No.: 18/561,321

(22) PCT Filed: May 17, 2022
(86) PCT No.: PCT/EP2022/063306

§ 371 Date: Nov. 16, 2023

(30) Foreign Application Priority Data
May 19, 2021 (EP) 21174687.0

(51) Int. Cl. G16B 30/10; C12N 15/10; C12Q 1/6834; G16B 40/20; G16B 45/00

(52) U.S. Cl. CPC G16B 30/10; C12N 15/1096; C12Q 1/6834; G16B 40/20; G16B 45/00

(57) ABSTRACT

The present invention relates to a computer-implemented analysis of spatial abundance of poly-A containing RNA in a tissue sample, comprising the steps of: (i) obtaining imaging data and the sequencing data, (ii) registering the imaging data and detecting the beads, and employing a first machine learning method to obtain a first barcode set from the imaging data, (iii) processing the sequencing data to obtain a second barcode set from the sequencing data, (iv) processing the first and second barcode sets by an optimal transport framework and/or supervised machine learning to match the data sets to each other and to obtain matched barcodes, (v) outputting based on the matched barcodes a matrix holding the expression values for each gene that was identified in each bead found in the data.

The present invention relates a method and system for 3D reconstruction of tissue gene expression data. In particular, the invention relates to a system and method for 3-dimensional (3D), reconstruction of a tissue gene expression or transcriptome data, their respective visualization and analysis.

A 3D visualization and analysis of genome-wide gene expression patterns of tissue samples can be used in several biomedical applications, for instance in the investigation of metastasis processes in tumor tissue. Investigation of gene expression profile of diseased tissues has been extensively performed in the past and is central for basic understanding of disease mechanisms.

It is particularly important for the identification of novel drug targets and biomarkers to improve patient outcomes. Unbiased investigation of all genes in parallel is essential to find novel therapeutic entities and can only be achieved through Next Generation Sequencing, NGS, technology. Since tumor and other diseased tissues are composed of various cells, each with distinct contributions to a disease, methods for analysis of gene expression at single cell resolution are key to discern molecular mechanisms.

In an other aspect, cells are arranged in space, which is important to understand for instance the interplay of immune and cancer cells, which a central topic in immuno-oncology. Methods have been developed to investigate expression of all genes at single cell resolution and in 2D tissue sections, see Rodriquez et al., Science (2019) 363, 1463-1467; Vickovic et al. Nat Methods (2019) 16, 987-990.

Since a tumor tissue is three-dimensional, 2D measurements are insufficient to capture its heterogeneous nature. Therefore, this invention extends and advances the presented concepts through a) improved methods for generation of 2D gene expression “images” and b) novel methods for constructing 3D representations from 2D gene expression images. These 3D gene expression “images” can be visualized for researchers to investigate expression of genes of interest, but can also be analyzed as integrated dataset to identify novel disease mechanism, targets, and biomarkers.

Genome-wide expression analysis at high spatial resolution using the so-called Slide-Seq technology has been described (Rodriquez et al., Science 363, 1463-1467 (2019); WO 2019/213254 A1). In the Slide-seq method, uniquely DNA-barcoded 10-mm microparticles (“beads”) are packed onto a rubber-coated glass coverslip to form a monolayer termed a “puck”.

Hereinafter the term “barcode” is used to when referring to a molecular tag for unambiguously identification of the microparticles.

These pucks are used to capture mRNA released from tissue cryosections. The captured mRNA is then analyzed using SOLID (sequencing by oligonucleotide ligation and detection) chemistry.

Genome-wide Spatial Expression Profiling in Formalin-fixed paraffin embedded (FFPE) Tissues is described in Villacampa et al. (https://doi.org/10.1101/2020.07.24.219758). The procedure takes advantage of well-established, commercially available methods for imaging and spatial barcoding using slides spotted with barcoded oligo(dT) probes to capture the 3′ end of mRNA molecules in tissue sections.

The present invention is directed at providing three-dimensional expression data. A general principle of the invention is the combination of data from a two-dimensional imaging technique, preferably a microscopy imaging, with data from a sequencing technique and subsequently to obtain a 3D visualization of said data. The method of the invention is thus based on a technique similar to the SlideSeq technology, i.e. using cryosections, and/or or the Villacampa et al. approach (loc. cit.), i.e. using FFPE sections, described herein above, however, the present invention in particular accounts for the three-dimensional nature of the samples.

Other suitable basic techniques that can be used in the context of the present invention include the high-definition spatial transcriptomics (HDST) (Vickovic et al., Nature Methods 16:987-990 (2019)) and the Visium technology (10× Genomics; e.g. WO 2012/140224 A1, WO 2014/060483 A1).

In general, for analysis of gene expression patterns of tissue samples, microscopy-based imaging of a puck may be used in combination with a sequencing technique. However, such a combination results in two different types of data:
(1) the imaging data, which is obtained when a puck, hereinafter also referred to as an “array structure”, is imaged in order to register the bead locations on it; and
(2) the sequencing data which quantifies gene expression in every bead.

Therefore, it is an object of the present invention to provide an improved method and system to match the two different types of data. Further objects are to provide a largely automated, spatially resolved, 3D visualization, and analysis of gene expression patterns of tissue samples.

The above objects, among others, are solved by the subject-matter of the independent claims. The dependent claims relate to further aspects of the invention.

A preferred aspect of the invention, relates to analyzing the gene expression on the mRNA level. However, in further embodiments, the invention is adapted for detecting spatial protein distribution in tissues by using oligonucleotide-tagged antibodies. For detection of proteins in tissue sections, the tissue sections are incubated with antibodies which are tagged with DNA barcodes composed of an identifier sequence, which is specific for each antibody and an oligo-A sequence. Upon incubation with antibodies and washing, the DNA tags can bind to the oligo-dT coated beads. The bound antibody DNA tags are also converted into a next generation sequencing library, which enables quantification of antibodies at each position of the puck, which serves as a proxy for protein counts at each position.

Hence, the present invention broadly relates to a method for analyzing spatial abundance of biomacromolecules, preferably RNA, DNA or protein, more preferably poly-A containing RNA such as mRNA, in a tissue sample from a subject, comprising the steps of:
(a) providing a plurality of consecutive sections, preferably cryosections, of said tissue sample,
(b) producing a plurality of array structures (“pucks”) by depositing for each array structure beads with an average diameter of from 1 to 100 μm on a solid support, wherein each bead comprises at least 1000 attached oligonucleotides and wherein each of the at least 1000 attached oligonucleotides of each bead comprises:
   (i) a bead identification sequence that is common to all at least 1000 oligonucleotides on each bead and that is unique to each bead in the respective array structure, and
   (ii) a biomacromolecule specific capture sequence, preferably an oligonucleotide sequence such as a poly-T sequence, to capture the biomacromolecules in said sample,
(c) identifying for each array structure the bead identification sequence and associated two-dimensional position on the solid support of individual beads of the beads deposited on the solid support by performing a sequence-by-synthesis technique using a microscope,
(d) contacting each array structure of the plurality of array structures with a section of the plurality of sections of said tissue sample and permeabilizing the tissue sections, thereby capturing the biomacromolecules in said sample by said capture sequences attached to the beads,
(e) identifying for each array structure the biomolecules bound to the oligonucleotides of the beads and the associated bead identification sequence for each biomacromolecule to be identified,
(f) matching for each array structure the bead identification sequence determined in steps (c) and (e) wherein a two-dimensional position in the array structure is assigned to the identity (e.g. the sequence in the base of an RNA) of each captured biomolecule,
(g) aligning the two-dimensional sequence data obtained in step (f) for consecutive sections thereby obtaining spatially-resolvable biomacromolecule abundance data from the tissue sample, wherein said aligning comprises performing a transformation on one or more reference biomolecules (i.e. typically reference gene products such as mRNA or proteins) in the two-dimensional sequence data obtained in step (f) for consecutive sections.

The term transformation in step (g) refers to any computational method, preferably a method of computer vision, more preferably of image processing, which allows to align a series of consecutive sections into three dimensions. This may comprise Scale Invariant Feature Transform (SIFT) to identify all genes that agree on a common transformation between any pairs of sections (e.g. +/−2 in the z-direction). SIFT extracts corresponding interest points. This transformation is a rigid transformation and includes only translations and rotations.

The transformation may further comprise globally minimizing the distance between all corresponding image points of all consecutive sections, yielding a single rigid model for each section.

The transformation may further comprise, in a refinement step, using an Iterative Closest Point algorithm on all sequenced points, not interpreted as an image, where nearest neighboring points with a similar expression are assigned to be correspondences.

The transformation may further comprise using all of the above correspondences to globally solve and identify an affine transformation model for each section.

A very important application of this method is the analysis of spatial mRNA abundance. In this case, the biomacromolecule specific capture sequence is typically a sufficiently long poly-T sequence (typically DNA poly-T) to capture complementary poly-A strands of the mRNA. Such a method is described below in much detail. However, when the spatial abundance of a protein or a plurality of proteins is to be analyzed, (monoclonal) antibodies against the protein(s) of interest can be used, wherein these antibodies are conjugated to poly-A oligonucleotide sequences (preferably DNA) complementary to the poly-T sequences (preferably DNA) on the beads. If a plurality of different proteins is to be analyzed, the oligonucleotide sequence conjugated to the antibodies (comprising the poly-A sequence) also includes a unique identification sequence for each antibody of a certain specificity. Hence, the protein(s) of interest will be captured by an antibody which itself will be captured by the poly-T sequences on the bead. Sequencing can then link the bead identification sequence with the identification sequence of the antibody.

The reference biomolecules of step (g) are typically reference genes, or more precisely the products of reference genes (mRNA or proteins depending on the biomolecules the abundance of which is to be analyzed) which have a relatively high abundance (i.e. expression) and variability among/within tissues and are therefore suitable to provide additional spatial information. The reference genes/gene products may also be called “gene of high information”. Examples include the genes eve, ftz, twi and zen in the Drosophila embryo or the Purkinje cells marker genes Pcp4 in mouse brain cerebellum.

Hence, in a particularly prominent aspect, the present invention relates to a method for analyzing spatial abundance of poly-A containing RNA in a tissue sample from a subject, comprising the steps of
(a) providing a plurality of consecutive sections, preferably cryosections, of said tissue sample,
(b) producing a plurality of array structures by depositing for each array structure beads with an average diameter of from 1 to 100 μm on a solid support,
wherein each bead comprises at least 1000 attached oligonucleotides and wherein each of the at least 1000 attached oligonucleotides of each bead comprises:
   (i) a bead identification sequence that is common to all at least 1000 oligonucleotides on each bead and that is unique to each bead in the respective array structure, and
   (ii) a poly-T sequence to capture mRNA molecules in said sample,
(c) identifying for each array structure the bead identification sequence and associated two-dimensional position on the solid support of individual beads of the beads deposited on the solid support by performing a sequence-by-synthesis technique using a microscope,
(d) contacting each array structure of the plurality of array structures with a section of the plurality of sections of said tissue sample and permeabilizing the tissue sections, thereby capturing poly-A containing RNA in said sample by said oligonucleotides attached to the beads,
(e) sequencing for each array structure the RNA molecules bound to the oligonucleotides of the beads and the associated bead identification sequence for each RNA sequenced,
(f) matching for each array structure the bead identification sequence determined in steps (c) and (e) wherein a two-dimensional position in the array structure is assigned to the nucleotide sequence of each captured RNA,
(g) aligning the two-dimensional sequence data obtained in step (f) for consecutive sections thereby obtaining spatially-resolvable RNA abundance data from the tissue sample, wherein said aligning comprises performing a transformation on one or more reference of poly-A containing RNAs in the two-dimensional sequence data obtained in step (f) for consecutive sections.

As stated herein above, in one preferred aspect, the poly-A containing RNA is mRNA.

The samples may be from any tissue. However, particular sample types include samples selected from the group consisting of brain, heart, kidney, liver, lung and pancreas. The samples may for example be samples from cancer tissue. The subject herein is typically a mammal, preferably a human. For example, cancer patients are particularly relevant patients for applying the method of the present invention.

The tissue may be permeabilized by trypsin, collagenase and/or other enzymes after step (d). In certain aspects, the tissue is permeabilized by trypsin, collagenase and/or other enzymes after step (d).

The solid support may particularly be a rectangular glass slide or sticky tape. The solid support may typically have a diameter of from 0.1 to 100 mm. In a preferred aspect, the diameter is 1 to 100 mm, more preferably 1 to 40 mm, even more preferably from 1 to 10 mm. An exemplary diameter is about 3 mm. Typically, the solid support is an adhesive plastic or glass surface or Polydimethylsiloxane, PDMS, matrix. It can also be acrylic glass. Bead attachment on glass can be facilitated using silicone or other glues/adhesives.

The beads advantageously form a monolayer on the solid support. Typically, each array structure comprises of from 10,000 to 10,000,000 beads, preferably 50,000 to 200,000 beads, more preferably about 100,000 beads. The beads may comprise polystyrene, glass, polymethacrylate and/or polyacrylamide. Hence, in particular aspects, the beads are polystyrene, Poly(methyl methacrylate), PMMA, or glass beads.

The beads typically have an average diameter of from 1 to 30 μm. In a preferred aspect, the beads have an average diameter of from 1 to 10 μm, more preferably 10 μm. Each bead typically comprises between 1×10³ to 1×10⁹ attached oligonucleotides, preferably between 1×10⁵ and 1×10⁸ attached oligonucleotides, more preferably between 1×10⁷ and 1×10⁸ attached oligonucleotides, even more preferably about 3×10⁷ attached oligonucleotides.

The attached oligonucleotides are most typically DNA oligonucleotides. They comprise a bead identification sequence that is common to all at least 1000 oligonucleotides on each bead and that is unique to each bead in the respective array structure, and a poly-T sequence to capture mRNA molecules in said sample. The oligonucleotides may further comprise a unique molecular identifier, UMI, sequence, preferably having a length of from 6 to 12 nucleotides, preferably 8 nt. The bead identification sequence typically has a length of from 4 to 20 nucleotides, preferably 12 nt. The oligonucleotides may further comprise a linker sequence and/or one or more primer hybridization sequence(s). Hence, in some aspects sequencing in step (c) comprises contacting the array structure with primers. The primers may be DNA primers or LNA primers.

In certain aspects, sequencing of the RNA molecules in step (e) comprises reverse transcription to obtain cDNA attached to the oligonucleotides of the beads and sequencing the cDNA molecules by a NGS technique. The NGS technique preferably is Sequencing-by-Synthesis, SBS, such as Illumina dye sequencing.

In a preferred aspect of the method of the invention, in step (f) an Optimal transport Problem based approach is used and/or in step (g) a Scale-Invariant Feature Transform algorithm is used.

The present invention also pertains to the use of the method of the present invention for gene expression profiling of a tissue sample.

The invention further relates to a method for the computer-implemented analysis of spatial abundance of biomolecules, particularly poly-A containing RNA, in a tissue sample, comprising the steps of:
i) obtaining the imaging data and the sequencing data in a method as described herein above,
ii) registering the imaging data and detecting the beads, and employing a first machine learning method to obtain a first barcode (i.e. a molecular tag) set from the imaging data,
iii) processing the sequencing data to obtain a second barcode set from the sequencing data,
iv) processing the first and second barcode sets by an optimal transport framework and/or supervised machine learning to match the data sets to each other and to obtain matched barcodes,
v) outputting based on the matched barcodes a matrix holding the expression values for each gene that was identified in each bead found in the data.

Hence, the invention relates to a method for the computer-implemented analysis of spatial abundance of poly-A containing RNA in a tissue sample, comprising the steps of:
i) obtaining
   (i1) imaging data of a plurality of consecutive sections of said tissue sample, and (i2) two-dimensional sequencing data of said poly-A containing RNA in said sections, preferably two-dimensional quantitative gene expression data, preferably in a method according to any one of claims 1 to 10,
ii) registering the imaging data and detecting the two-dimensional position of beads in said imaging data, and employing a first machine learning method to obtain a first barcode set from the imaging data,
iii) processing the two-dimensional sequencing data to obtain a second barcode set from the sequencing data,
iv) processing the first and second barcode sets by an optimal transport framework and/or supervised machine learning to match the data sets to each other and to obtain matched barcodes,
v) outputting based on the matched barcodes a matrix holding the expression values for each gene that was identified in each bead found in the data.

The method of may further comprise the step of visualizing the output in a three-dimensional representation of said tissue sample.

In a related aspect, the invention pertains to a data processing system comprising means for carrying out the steps of the method for the computer-implemented analysis of spatial abundance of biomolecules, particularly poly-A containing RNA in a tissue sample.

In a further related aspect, the present invention pertains to a computer program product comprising instructions which, when the program is executed by a computer, cause the computer to carry out the steps of the method for the computer-implemented analysis of spatial abundance of biomolecules, particularly poly-A containing RNA in a tissue sample.

Further, the invention pertains to a computer-readable storage medium comprising instructions which, when executed by a computer, cause the computer to carry out the steps of the method for the computer-implemented analysis of spatial abundance of biomolecules, particularly poly-A containing RNA in a tissue sample.

It is a core concept of the invention that since the sequence data of the bead barcodes are determined by both microscopic imaging and sequencing, possible errors of the respective methods are improved by, among other things, a matching process.

In a general aspect of the invention, for spatial gene expression analysis of cryopreserved tissue sections, arrays with special particles and/or beads are prepared, hereinafter referred to as “puck array” or “array structure”. In particular, these beads carry oligo(dT) oligos labeled with DNA barcodes, which are later used to identify corresponding gene sequences of the biological tissue section in a position-specific manner, i.e. bead-dependent.

In a general aspect of the invention, by means of a microscope, the tissue sections to be examined as well as fluorescence images of the prepared bead arrays are imaged and the individual particles and/or beads are decoded, preferably according to the different recorded fluorescence spectra, this is hereinafter referred to as “in situ indexing”.

In a general aspect of the invention, a correction approach is based mathematically on the so-called optimal transport theory. Based on the correction approach the best possible matching of the two data sets is determined. Additionally, or alternatively supervised machine learning, preferably recurrent neural networks, are used for correction. It has been found that machine learning works much better, i.e. a better match between the two datasets is obtained.

In a general aspect of the invention, the correction is followed by a combination of the obtained expression data with the associated spatial information, i.e. 2-dimensional information, obtained from the bead barcodes. Barcode sequences in this context are unique identifier nucleic acid sequences that allow identification of the respective beads, herein also referred to as “bead identification sequence”. The barcode sequences are up to 20 nt in length, preferably between 4 to 20 nt, typically 12 nt long. They can comprise multiple segments. Unique molecular identification, UMI, nucleic acid sequences which identify the input nucleic acids can optionally be used in addition. They typically have a length of from 6 to 12 nucleotides, preferably 8 nt.

In a general aspect of the invention, an image registration for final 3D reconstruction of the tissue images with the integrated gene expression data is performed using the “optimal transport theory”, preferably in combination with other procedures.

In a general aspect of the invention, to determine a gene expression, the tissue sections are fused with the bead arrays so that polyadenylated mRNA is bound and a gene expression can be quantified using known methods. In a preferred embodiment of the invention, this also involves sequencing of the particle barcodes, preferably using a NGS method, more preferably the Illumina sequencing method.

It is a general advantage of the invention, that with the correction and registration method, a high degree of automation can be achieved. Furthermore, the use of specific software, including known artificial intelligence methods, can further improve the degree of automation in sub-steps of the process.

### BRIEF DESCRIPTION OF THE DRAWINGS

The above-described objects, advantages and features of the present invention, as well as others, will be more readily understood from the following detailed description of certain preferred embodiments of the invention, when considered in conjunction with the accompanying drawings in which:

FIG. 1 shows a schematic drawing of a microscope setup 130 according to an embodiment of the invention;

FIG. 2a shows an overview of a workflow for generation of 3D molecular tissue models according to an embodiment of the invention;

FIG. 2b shows an overview of a library preparation protocol according to an embodiment of the invention;

FIG. 3 shows a flow chart of a pipeline for computational analyses according to an embodiment of the invention;

FIGS. 4a to 4c show a schematic representation of the optical sequencing data 304 obtained from pucks 120 at different subsequent serial cryosections representing different z-sections according to an embodiment of the invention.

FIGS. 5a to 5c show a schematic representation of sequencing data 402 of obtained from sequencing according to an embodiment of the invention;

FIG. 6 shows a schematic representation of an alignment of the spatial transcriptomics datasets according to an embodiment of the invention;

FIG. 7 shows a flow chart of the alignment of the spatial transcriptomics datasets according to an embodiment of the invention;

FIG. 8 shows a flow chart of the alignment of the spatial transcriptomics datasets according to an embodiment of the invention including preferred optional steps;

FIG. 9a: shows a gene expression on a 2D section of brain cerebellum and for the gene Pcp4 according to an embodiment of the invention. Each bright spot represents a position on the puck where a bead is located and where the Pcp4 is expressed; and

FIG. 9b: shows a 3D representation of a Drosophila embryo with two genes highlighted according to an embodiment of the invention; eve and ftz. Each location that is bright represents a true location of a cell in the embryo and expresses either eve or ftz.

### DETAILED DESCRIPTION OF THE INVENTION

In the following, exemplary embodiments of the invention will be described. It is noted that some aspects of any one of the described embodiments may also be found in some other embodiments unless otherwise stated or obvious. However, for increased intelligibility, each aspect will only be described in detail when first mentioned and any repeated description of the same aspect will be omitted.

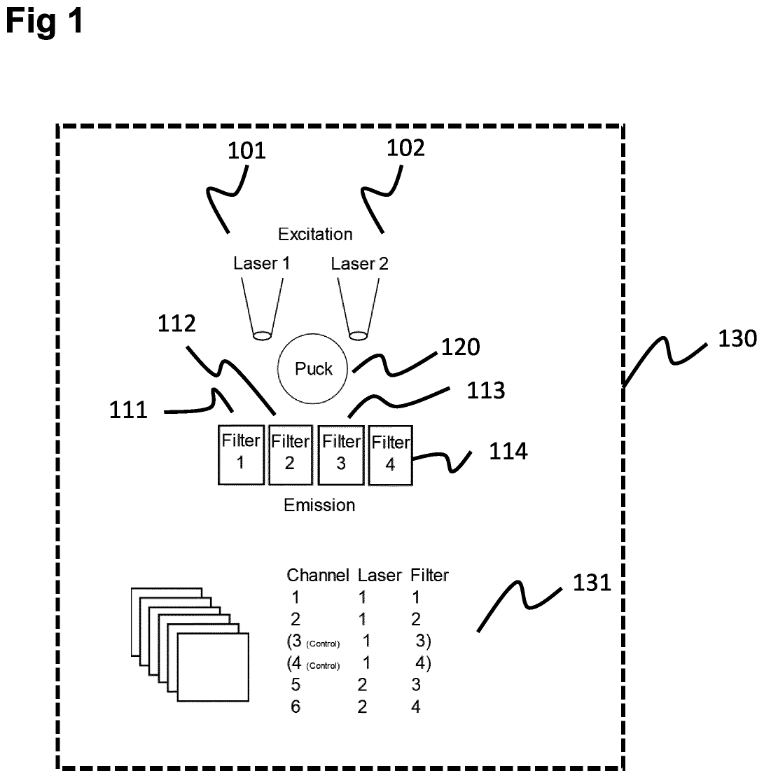

FIG. 1a shows a schematic drawing of a microscope setup 130 according to an embodiment of the invention. Two lasers 101 and 102 are used to excite fluorescent nucleotides on the puck 120. The emission profiles are collected through four filters 111, 112, 113, 114 corresponding to optimal emission characteristics of respective fluorophores. The intensity profiles are recoded in 4 channels plus 2 control channels 131 where excitation/emission settings should not lead to detectable signal.

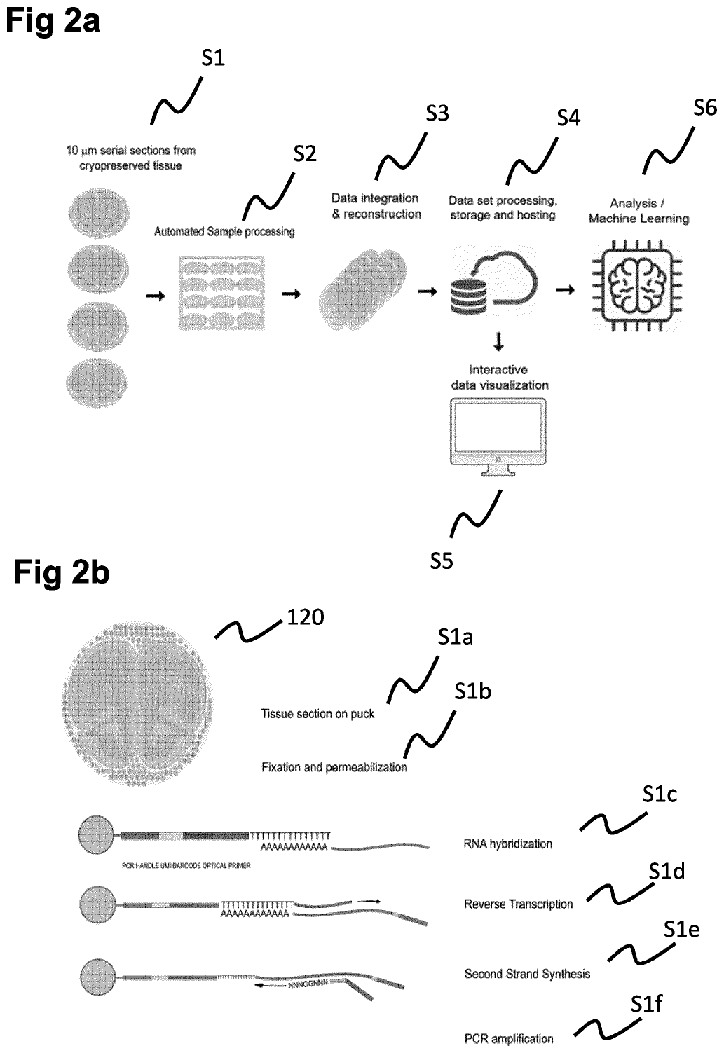

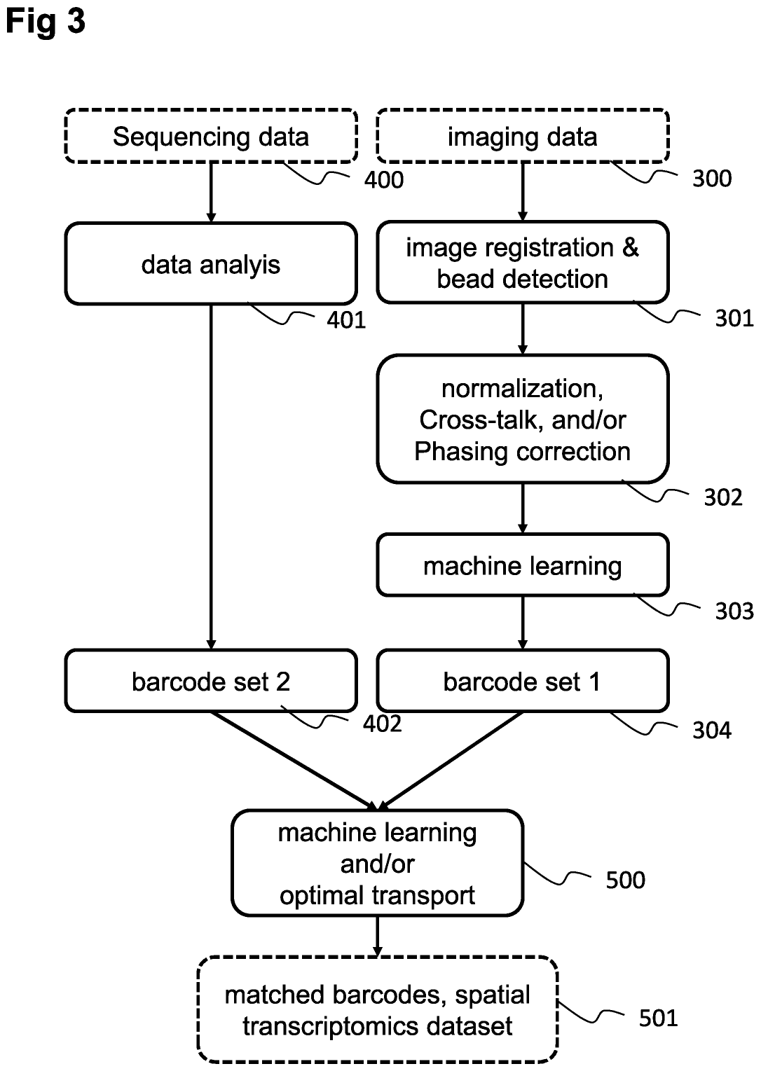

FIG. 2a shows an overview of a workflow for generation of 3D molecular tissue models according to an embodiment of the invention. Serial cryosections of a tissue are placed onto pucks in and processed into next generation sequencing libraries resulting in 2D representations of gene expression in step S1. This process is preferably automated in step S2. 2D tissue models are computationally integrated into 3D tissue models of gene expression in step S3. Datasets are stored in databases in step S4 with interfaces for data visualization in step S5 and downstream analysis in step S6, preferably by machine learning algorithms.

FIG. 2b shows an overview of a library preparation protocol according to an embodiment of the invention. The tissue sample is placed on pucks 120 in step S1a, fixed and enzymatically digested to release RNA in step S1b. Polyadenylated RNA hybridizes to oligo-dT DNA capture sequences attached to beads on the puck surfaces in step S1c. RNA is then reverse transcribed in step S1d, and a second strand is synthesized using a random DNA primer in step S1e. Resulting second strands are PCR amplified in step S1f and sequenced by NGS.

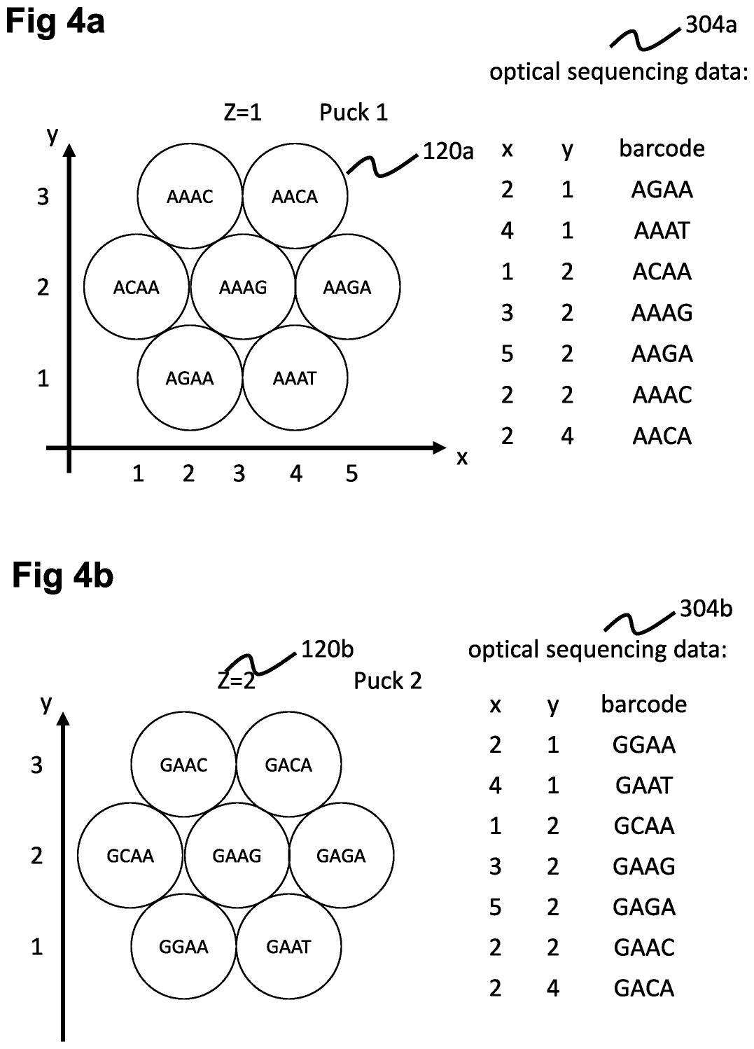

FIG. 3 shows a flow chart of a pipeline for computational analyses according to an embodiment of the invention. On the right: the imaging data 300 collected via the microscopy is analyzed to detect the beads on the puck and to register the images of the different optical sequencing cycles 301. This data is further processed 302, further processing comprises one or more of normalization, cross-talk and/or phasing correction. Supervised machine learning 303 is preferably used for basecalling to obtain a first barcode set 304. On the left: the data obtained through sequencing 400 is processed 401, preferably processed with standard data analysis tools, which generates a second barcode set 402. The first and second barcode sets 304 and 402 are then compared to one another and matched, preferably via supervised machine learning and/or optimal transport methods, resulting in a matched barcodes set 501.

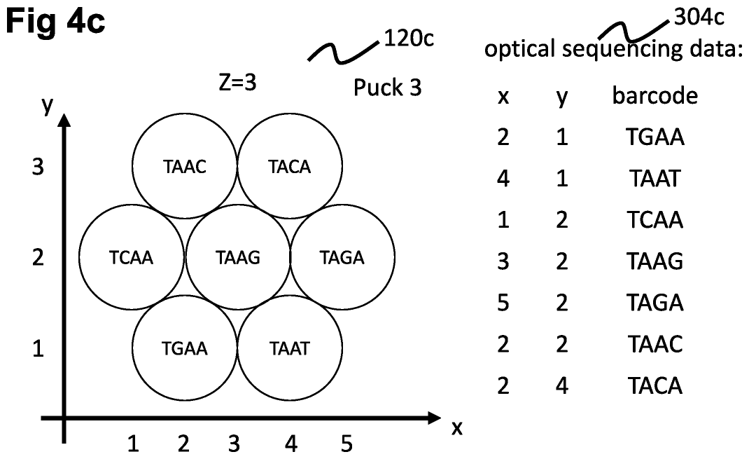

FIGS. 4a to 4c: show a schematic representation of the optical sequencing data 304 obtained from pucks 120 at different subsequent serial cryosections representing different z-sections according to an embodiment of the invention. That is, FIG. 4a shows the representation of a first puck 120a at a position z=1 and the resulting optical sequencing data 304a; FIG. 4b shows the representation of a second puck 120b at a position z=2 and the resulting optical sequencing data 304b; and FIG. 4c shows the representation of a third puck 120c at a position z=3 and the resulting optical sequencing data 304c. The puck is represented as a circular disk and the beads have been detected on it and are represented with a 4 digit barcodes. These may be obtained by sequentially reading out ACGT at the first 4 positions. The figures illustrates basecalling where the nucleotides of the bead barcodes are determined after data analysis.

In a further step the tissue samples are placed on the pucks. Each tissue slice equals one z position. Next the preparation of cDNA next generation sequencing libraries is performed and sequencing of cDNA libraries containing bead DNA sequences is performed.

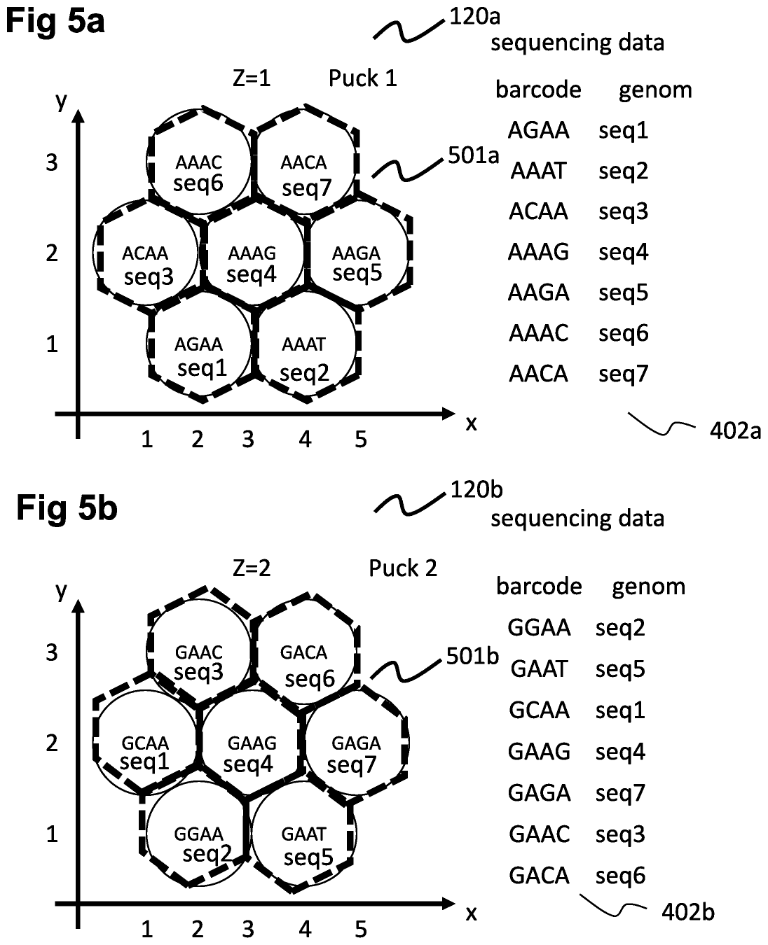

FIGS. 5a to 5c show a schematic representation of sequencing data 402 of obtained from sequencing according to an embodiment of the invention. Given that the barcodes of the pucks have been obtained, cf. FIGS. 4a to 4c, now the data of the two data sets can be matched based on matching barcodes. In other words, specific positions on a specific puck can be matched with specific sequencing data comprising corresponding barcodes. The result is a spatial transcriptomics dataset 501. Every dataset is a puck 120a, 120b, 120c, with the beads registered in space and containing gene expression counts for each bead. For each gene, going in the z-direction, these datasets can be regarded as similar, but distorted images, e.g. rotated, translated, and/or stretched. In FIGS. 5a to 5c the datasets are clockwise rotated by 45 degree from FIG. 5a to FIG. 5b and by 180 degree from FIGS. 5a to 5c.

In a preferred embodiment of the invention, the matching 500 of the optical sequencing data 304 with the sequencing data 402 is performed by first matching beads if they have the same barcode sequence, e.g. ACGTAGTACG==ACGTAGTACG. Second, machine learning is used to correct the bases called in optical sequencing data. Preferably the machine learning model is trained on the matches of the optical sequencing data 304 with the sequencing data 402, and the run on the barcodes of the optical sequencing data that do not match any of the barcodes of the sequencing data. This enhances the number of matches significantly. Third, optionally, optimal transport methods are used to find the optimal matching between the remaining barcodes of the optical sequencing data 304 with the sequencing data 402.

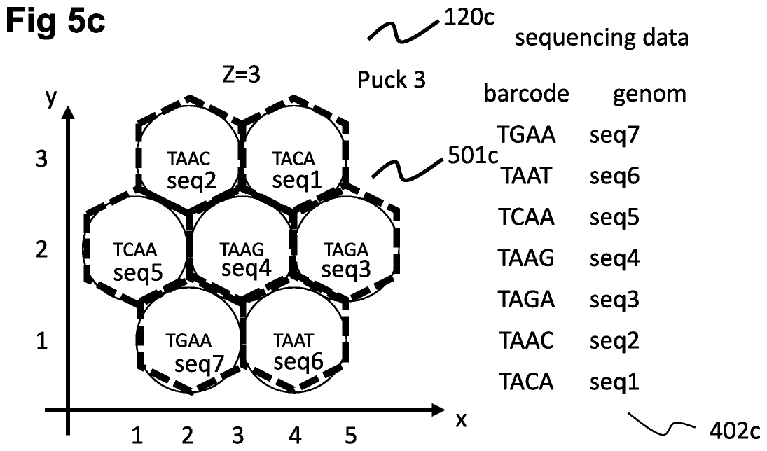

FIG. 6 shows the alignment of the spatial transcriptomics datasets 501a, 501b, 501c based on the genomic information using SIFT 600 and the 3D reconstruction 700 of the z slices based on the aligned puck images.

In a preferred embodiment of the invention, The SIFT alignment 600 is performed on the gene expression data to this end the gene every gene on the puck can be regarded as an image and the alignment is performed on all genes simultaneously and finds the optimal transformation to align the pucks.

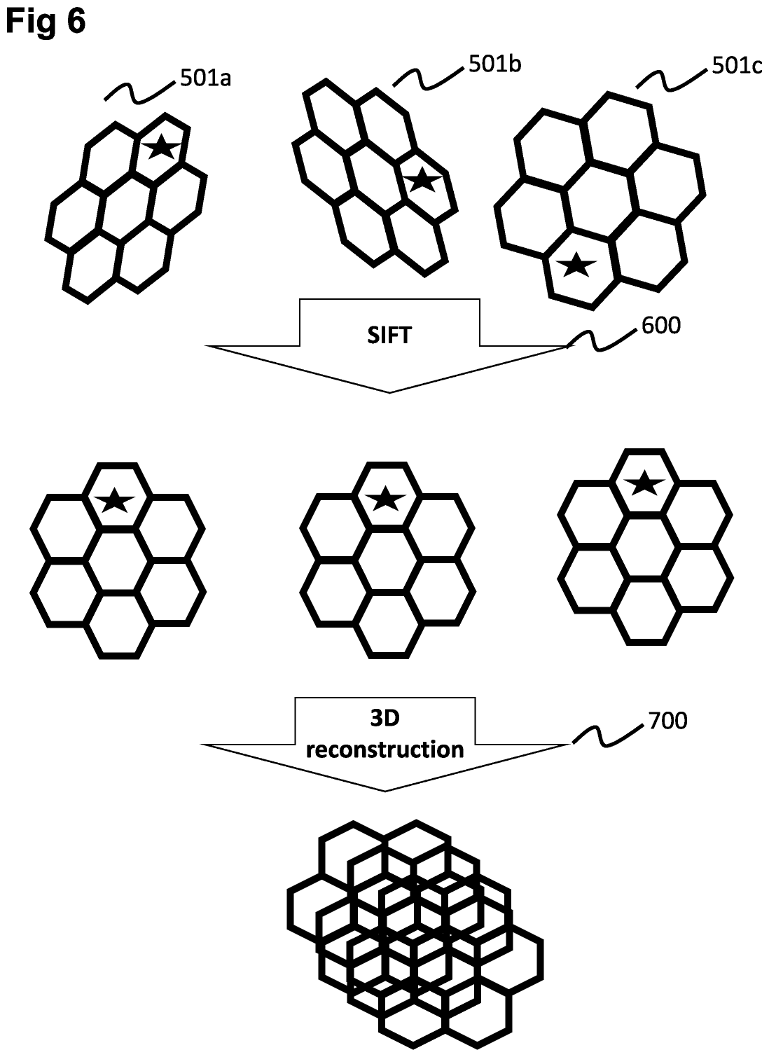

FIG. 7 shows a flow chart of the alignment of the spatial transcriptomics datasets according to an embodiment of the invention. The alignment comprises a preprocessing S3a, a data access S3b, and an image alignment S3c.

In a preferred embodiment, the preprocessing S3a comprises collecting 801 spatial transcriptomics datasets 501, preferably in a CSV-File-based format for a single puck (2D) or multiple pucks (3D). In a preferred embodiment, the preprocessing S3a further comprises Log-normalization 802 of the collected datasets. In a preferred embodiment, the preprocessing S3a further comprises a conversion 803 of datasets to N5 format, which allows for parallel write, cached, fast, and/or partial access.

In a preferred embodiment, the data access S3b comprises an access 804 through ImgLib2 and to render each gene as an image using Gaussian distributions based on random sample locations. In a preferred embodiment, the data access S3b further comprises an access 805 of individual expression values and locations.

In a preferred embodiment, the image alignment S3c comprises pairwise alignment of all pucks (z+−2) using SIFT 600. In a preferred embodiment, the image alignment S3c further comprises solving 601 globally optimal alignment transforms across all pucks. In a preferred embodiment, the image alignment S3c further comprises saving 602 the transformations to N5, which is preferably used for all subsequent operations. In a preferred embodiment, the image alignment S3c further comprises refining 602 the pairwise alignment of all pucks (z+−2) using an Iterative Closest Point Algorithm.

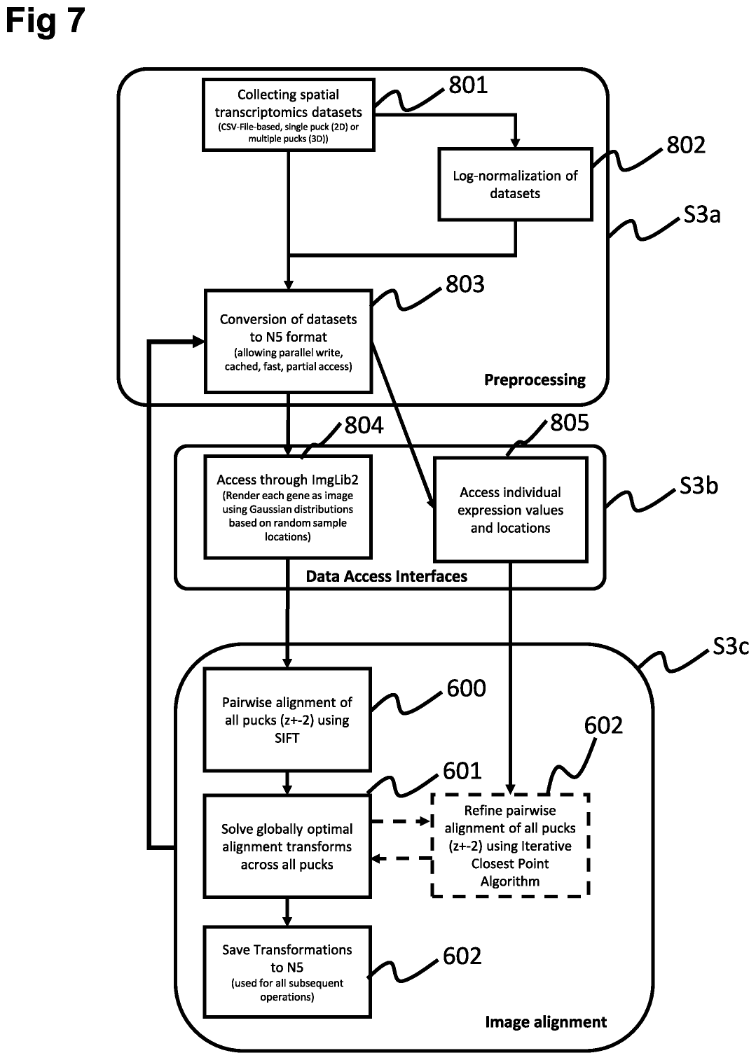

FIG. 8 shows a flow chart of the alignment of the spatial transcriptomics datasets according to an embodiment of the invention including preferred optional steps. The additional optional steps comprise one or more of postprocessing S3d, an expression intensity adjustment S3e, a visualization/quality control S5, and downstream processing and scientific analysis S6.

In a preferred embodiment, the postprocessing S3d comprises applying image filters for smoothing, e.g. median, filter single spots.

In a preferred embodiment, the expression intensity adjustment S3e comprises globally minimizing a distance between all puck expression levels across all pucks and/or saving the transformations to N5, which used for visualization.

In a preferred embodiment, the downstream processing and scientific analysis S6 comprises providing an access via Java and/or Python.

In a preferred embodiment, the visualization/quality control S5 comprises an interactive display of 2D/3D data using a BigData Viewer and/or rendering 2D/3D data as TIFF images.

### I. Experimental Setup

As detailed above FIG. 1 shows the microscope setup according to an embodiment of the invention. In a preferred embodiment of the invention, the microscope setup 100 is equipped with two excitation lasers 101 and 102 and four emission filters 111, 112, 113, and 114, preferably adjusted to the emission spectra of the fluorescent nucleotides, preferably to the spectral of the fluorescent nucleotides contained in the extension reagent mix, used in the Sequence-by-Synthesis reactions. Imaging is performed using distinct combinations 130 of excitation laser and emission filter setting which excite the specific fluorophores attached to each of the four types of DNA nucleotides, i.e. A, T, G, or C, used for later decoding of the bead DNA barcodes.

The emission filters are preferably configured to record emission intensities for the maximum emission bandwidth of the respective nucleotides. For instance, excitation laser 1 and emission filter 2 may encode an adenosine, A, base. Imaging can be performed in 4 channels, i.e. excitation 1-emission 1, excitation 1-emission 2, . . . etc. or 6 channels, where 2 channels serve as internal control, since excitation laser 1 should not produce emission of the fluorescent nucleotides which emit within bandwidth of filter 4. Since some nucleotide combinations have overlapping excitation and emission spectra, computational correction for spectral overlap may be necessary, cf. spectral unmixing below.

The microscope setup hence produces two-dimensional images with 4 or 6 channels corresponding to the excitation/emission properties of fluorescent bases. Furthermore, imaging in Z direction may be performed optionally as well in order to correct for differences in focus area. Additionally, tile scans of several images may be performed optionally in order to capture larger areas of the imaged object, i.e. the puck.

### Optical Decoding

In an embodiment of the invention, the spatial gene expression profiling requires a registration of the spatial coordinates of the bead particles within the pucks. To this end, an oligonucleotide primer is annealed to the optical sequencing handle, which is present on all oligos on each bead.

In 12 subsequent cycles, a randomized DNA cell barcode, which is unique to each bead, is decoded in a sequence by synthesis reaction. Preferably, similar to Illumina systems, at least one fluorescently labeled nucleotide complementary to the respective base in the cell barcode is incorporated at the end of the hybridized DNA primer.

In a preferred embodiment of the invention, said nucleotides are chemically modified, such that a chemical group blocks the incorporation of further nucleotides. This reaction is performed using a DNA polymerase and fluorescent nucleotides with a chemical blocking group at the 3′ hydroxy group of the nucleotide (extension reagent mix).

For each bead particles the obtained fluorescence spectra are recorded. The spectra can be recorded in 4 or 6 channel images, optionally across several focus areas (Z-direction) and as tile scans (s. above). The blocking groups of each nucleotide are chemically removed, and the fluorescent dyes are cleaved such that further fluorescent nucleotides can be incorporated. This step is performed using reagents such palladium catalysis (unblocking reagent mix).

In an embodiment of the invention, this process is repeated such that the complete cell barcode for each bead is decoded, preferably repeated 12 times. In a preferred embodiment of the invention, the process is performed with extension reagent mix and unblocking reagent mix.

After performing the optical decoding the cell barcodes of each bead particle are decoded in context of the beads' spatial positions, see FIGS. 4a to 4c.

In order to decode the spatial position of each bead DNA barcode on the puck, bead specific DNA barcodes are decoded using Sequence-by-Synthesis chemistry, which is performed in-situ using a specifically configured microscope setup.

In order to perform Sequence-by-Synthesis reactions, first a custom sequencing primer, preferably an LNA sequencing primer, is hybridized to the optical sequence handle on each oligo attached to the beads in an annealing reaction.

Once the LNA primer is hybridized, the extension reagent mix is added, containing fluorescent nucleotides as well as polymerase. Fluorescent nucleotides 3′ ends are blocked such that only single bases are incorporated in each round.

After fluorescent incorporation, the pucks are washed and scanned using the above microscope setup.

A 4 or 6-channel image is recorded using difference excitation/emission settings of the microscope. Additionally, the images can be recorded as z-stack or several images of adjacent reads of the puck can be imaged (tile scan). The fluorescent moieties are then cleaved using the unblocking mix. In the same step nucleotides are deblocked, which enables another round of nucleotide incorporation, imaging and deblocking.

These steps are repeated 12 times. Images from each sequencing cycle are processed as outlined in the computational methods.

### II. Sample Processing

In an embodiment of the invention, 10 μm polystyrene beads are used, e.g. are purchased from Chemgenes, Boston, MA. The beads are synthesized by Split-and-Pool synthesis (Macosko et al., Cell 161, 1202-1214 (2015)) in order to generate 12 nt DNA barcodes which are unique for all oligos attached to a bead but different between beads. Alternatively, unique DNA barcodes on each bead can be produced by a combinatorial indexing approach. For this, a bead pool with a constant DNA sequence is distributed over a large number of the pool.

A unique barcoding DNA oligo is added to each pool, which is composed of a constant DNA sequence complementary to the sequence originally present on the beads, and a unique DNA sequence with known sequence which differs between each pool. The barcoding oligo is hybridized to the beads and extended in a primer extension reaction using a polymerase or by ligation using a ligase. The beads are then pooled again and distributed over several wells again, and the procedure is repeated.

The number of unique barcodes of the resulting bead pools is the product of the number of barcoding oligos in each round (e.g. 96×96 barcodes=9216 barcodes). Importantly the resulting barcode sequences are not random but follow the combinations of the initial barcoding oligo pool, as opposed to the Split-and-pool procedure, which is advantageous for later correction of errors during the in situ indexing procedure.

The oligos attached to each bead preferably comprise:
a) a linker sequence attached to the polystyrene surface;
b) a PCR handle sequence, for amplifying the libraries, i.e. a so called “SMART” sequence;
c) an optional Unique Molecular Identifier, which is a degenerate 8 nt DNA sequence unique for each oligo, for eliminating PCR bias after next generation sequencing;
d) a bead specific 12 nt DNA barcode, which is preferably a 12 nt sequence and is identical for all oligos on one bead, but is different between beads.

e1) an optical sequencing handle, preferably a DNA sequence for hybridization of a (LNA) primer for optical sequencing; or
e2) alternatively a combination of 2 or more DNA barcodes are generated during the combinatorial barcoding procedure, each next to a specific optical sequencing handle, e.g. two 6 nt barcode segments which each two 20 nt optical sequencing handles, and
f) an oligo(dT) stretch, which captures mRNA molecules, preferably an oligo(dT) sequence for hybridization of polyadenylated RNA.

Pucks are produced by mixing a bead suspension, i.e. beads in TE buffer, with ethanol. The suspension is applied to an adhesive film and dried. About. 80.000 bead particles are added per pucks and form a diameter of ca. 3 mm. Pucks are then washed in water and air-dried as will be detailed below.

For production of microparticle arrays aqueous droplets of beads are placed on rectangular glass slides or adhesive films with a size of ca. 5 cm×2 cm. The droplets are either airdried and by default produce round puck structures with a size of 3 mm or are dried inside a silicone mask to produce pucks.

Pucks are then optically decoded as described above.

### Tissue Preparation and Illumina Library Generation

As detailed above FIG. 2b shows a schematic of a library preparation protocol according to an embodiment of the invention.

Fresh frozen biological specimens (of any kind) are embedded in embedding media, preferably a TissueTec OCT matrix, and frozen on dry ice. Embedded specimens are cut into 10 μm cryosections, preferably using a cryotome. Cryosections are then applied to optically decoded pucks attached to an adhesive film.

Cryosections and pucks are then fixed in 100% cold methanol several minutes, preferably for 5 to 15 minutes, most preferably for 10 minutes.

Fixed cryosections on pucks are rehydrated in PBS buffer and treated with collagenase mix for several minutes, preferably 15 to 25 minutes, most preferably for 20 min at about 37ºC.

Next, fixed sections are permeabilized using pepsin for about 10 to 15 min at 37° C. RNA from sections is released from the tissue during the permeabilization and hybridize to oligo(dT) sequences on beads.

In an embodiment of the invention, round plastic gaskets are placed on the pucks allowing for addition of small volumes, i.e. <100 μL, of reaction mixtures.

Hybridized RNA is the reverse transcribed using MaximaH-RTase for about 30 min at room temperature, RT, and 90 min at about 52° C. or 42° C. The step can be performed using the terminal transferase activity of the enzyme together with addition of another primer, which can be appended to the 3′ end of the cDNA in a template switch reaction. Tissue is then digested using proteinase K for about 60 min at 56° C.

The DNA-RNA hybrids synthesized during reverse transcription are denatured by adding 0.08M KOH and the RNA strand is washed away. A second DNA strand is synthesized by hybridizing a DNA oligo containing a random sequence and a PCR handle sequence to the first strand and extension by Klenow fragment polymerase for about 1 h at 37° C.

The cDNA libraries attached to the beads of each puck are then PCR amplified using a polymerase. For this step pucks are excised from the adhesive film and placed in PCR tubes.

Alternatively, second strand cDNA is eluted from the microparticle arrays using a basic solution. The eluate is used as input for PCR amplification.

The amplified cDNA libraries are then fragmented using a transposase. Fragmented libraries are again PCR amplified using Illumina Nextera reagents and sequenced on Illumina Next Generation Sequencers.

Alternatively, PCR amplicons are used directly as input for a second PCR step introducing sequencing adapters for multiplexing of several samples.

### Brief Summary of the Procedure

Pucks are produced by spotting a solution of ca. 80.000 beads on a silicone coated glass surface or an adhesive film. Liquid is dried to leave a solid puck with immobilized beads. DNA barcodes on beads are then optically decoded.

10 μm fresh-frozen cryostat tissue sections then placed onto the pucks. Tissue is fixed in and permeabilized in methanol and rehydrated. Pucks and tissue attached to adhesive film are placed into a gasket. Collagenase and pepsin are added for permeabilizing the tissue for efficient release of RNA. Polyadenylated RNA hybridizes to the oligo-dT sequences on the beads. The tissue is digested using a protease. cDNA attached to the pucks can then be amplified in a PCR reaction and processed into next generation sequencing libraries in a second PCR reaction. Optionally a second strand synthesis can be performed after the reverse transcription. In this case the cDNA second strand can be eluted from the puck before PCR amplification.

### Illumina Sequencing Data Processing

In an embodiment of the invention, the analysis of the Illumina sequenced data is performed with standard methods and tools that are widely available, e.g. bcl2fastq (software to convert raw data to genomic sequences), Drop-seq toolkit (a collection of tools that are used to manipulate high-throughput sequencing data), STAR (an aligner that maps sequenced reads to a genome), etc. Preferably, the input is paired-end reads, meaning two different parts of each captured molecule are sequenced. The first part, read1, contains the cellular barcode and potentially a unique molecular identifier, UMI, and the second part, read2, contains the molecule captured and is mapped against the genome.

The output is a large matrix with ˜100,000 cells (or beads) as columns and the quantified values in numbers of UMIs for all ˜20,000 genes (depending on the genome species) as rows.

### III. Data Processing

As detailed above FIG. 3 shows the data processing according to an embodiment of the invention. In detail, the imaging data, also referred to as optical sequencing data, 300 is registered and beads are detected 301, then the data is normalized, cross-talk and/or phasing is corrected 302. A first machine learning 303 is used to call bases, i.e. A, C, G or T, from the imaging data and to obtain a first barcode set 304. On the other hand, the sequencing data 400 is processed in a standard analysis 401 and a second barcode set 402 is obtained.

Both barcode sets 401 and 402 are then processed by an optimal transport framework and/or supervised machine learning 500 to match the data sets and to obtain matched barcodes 501, also referred to as spatial transcriptomics dataset.

### Image Data Acquisition

In a first step images from each cycle can be corrected with respect areas of focus and normalized.

The data analysis starts with acquiring microscopy images to decode the cell barcodes on the individual beads. This is done on a cycle-to-cycle basis. First the beads are identified from each microscopy image and their positions on the puck are stored.

The nucleotides of the optically sequenced cycle are identified for every bead on the puck. Subsequently the images from the cycles, preferably 12 cycles, are aligned and the raw fluorescent intensities for each bead and for each cycle is obtained.

In a preferred aspect, these two steps result in having the coordinates of every bead in 2 dimensions, as well as a 12×4 (12×6) matrix containing the raw intensities of the four (or six) channels for every cycle.

### Data Types

It is important to keep in mind that there are two sides and two different types of data:
(1) the imaging data 300, which is obtained when a puck 120 is imaged in order to register the bead locations on it; and
(2) the sequencing data 400, which quantifies gene expression in every bead.

The raw imaging data are microscope images of the pucks, preferably having 4, or 6, channels corresponding to the 4 bases A, C, G and T. After a data analysis the end product, i.e. the obtained data-set, preferably is a matrix with the locations of the beads and the associated barcode.

The raw sequencing data is in standard format, preferably “.fastq” files with paired-end short reads. Read 1 contains the barcode of every bead and a unique molecular identifier, UMI. Read 2 contains the sequence that will be mapped to the genome for quantifying the gene expression. The raw sequencing data is the input of the data analysis and the output is a matrix holding the expression values for each gene that was identified in each bead found in the data (like a single-cell gene expression matrix).

### Image Registration

The puck may be positioned slightly different in the microscope during the imaging of different cycles. This situation can cause rotation, translation, shear or scaling. Therefore, all the images of the imaging data need to be aligned to a reference frame. In a preferred embodiment, the image of the last cycle is used as the reference frame.

The steps of image registration comprise preferably one or more of:
i) A motion model is defined. Preferably this model is euclidean motion, which includes rotation and translation. Additionally, or alternatively, the model is an affine motion model that additionally includes shear and scaling. A motion model is basically a matrix of parameters for at least one of the rotation, the translation, the shear and the scaling.
ii) Iterating the registration. Preferably a pyramidal registration system is utilized in which the registration is in different resolutions. This helps to firstly register the coarser structure and then move to finer registration. For every level, a warping matrix is detected, i.e. the motion model parameters, by using the Enhanced Cross Correlation Maximization (Rodriquez et al., Science (2019) 363, 1463-1467) algorithm implemented in OpenCV1. This is an algorithm that is robust to illumination differences and uses the correlation coefficient between images for the optimization process.
iii) Transforming the overlay and channel images to the common reference frame. After calculating the warping matrix for every cycle, which is registered to the last cycle, this matrix is used to transform the overlay and channel images to the common reference frame.

### Background Correction

In a preferred embodiment of the invention, a morphological operations-based step is used to correct the background signal effects on the puck. Preferably a morphological opening with a 64×64 circle shaped kernel is applied to calculate the background image, and this is subtracted from the image to remove the background signal.

### Bead Identification

Bead detection is the process of detecting and segmenting the beads from microscopy images. For every nucleotide, i.e. cycle, of the barcode, there is a 6-dimensional microscopy image. Steps of the bead detection algorithm comprise:
i) Channel intensities are summed up to create a grayscale overlay image. This is the composite image that will be used for the bead detection.
ii) A median filter is applied to clean up the noise.
iii) As some images have low intensity levels that can hinder the accuracy of the bead detection, contrast limited adaptive histogram equalization, CLAHE, is used to increase the contrast, cf. Li, Lei, and Terence P. Speed. “An estimate of the crosstalk matrix in four-dye fluorescence-based DNA sequencing.” ELECTROPHORESIS: An International Journal 20.7 (1999): 1433-1442. This method works on small tiles of the image and prevents the over amplification of the contrast.
iv) The boundaries of the puck are detected by blurring the image, thresholding and calculating the biggest connected component which should correspond to the puck. Then, the objects or beads outside of the puck are filtered out.
v) To detect the beads, a Hough Circle transform algorithm (Massingham, Tim, and Nick Goldman. “All Your Base: a fast and accurate probabilistic approach to base calling.” Genome biology 13.2 (2012): R13) is used. Hough circle transform uses a two-step approach. Firstly, the edges in the image are detected, and secondly the pixels vote for the center and the radius of the possible circles. To use Hough transform, a few initial parameters are specified such as the approximate radius of expected beads and the thresholding parameters for the edge detection algorithm.
vi) After the application of the Hough transform and circle detection, bead centers and the estimated radius are acquired.
   Basecalling Algorithms from Optical Sequencing Data

In embodiments of the invention, different algorithms are used to transform the channel intensity signals to meaningful bases.

The challenge here is to process the raw intensities appropriately so that the correct A, C, G or T base is called for each of the cycles, preferably 12 cycles and for each bead.

In an embodiment of the invention, a probabilistic base calling is used. It can be assumed that original nucleotide sequences are confounded by two factors, namely spectral crosstalk and phasing.

Based on an estimate the interaction matrices for crosstalk and phasing, the inverse is used to correct the observed intensities and to acquire an estimation of the real intensities. This process includes two main steps: Estimating the crosstalk matrix and the phasing matrix.

The crosstalk estimation comprises the steps of:
i) Taking every pair of channels from the first cycle, and scatter plot the bead intensities. A first axis is channel A and a second axis is channel B. For the channels that have spectral crosstalk, the arms of the plot are not parallel to the respective axes. That is, when there is a high intensity for one channel, there may be some intensity in the other channel too.
ii) For the crosstalk matrix, every non-diagonal element corresponds to the slope of an arm. For every arm, the data points are received in a quantile range and the data points are binned.
iii) A line is fitted using simple linear regression to the binned data points to calculate the slope of the respective arm.
iv) This calculated slope is the value inserted into the crosstalk matrix and then the intensities can be transformed.

This process is continued iteratively, until the arms are parallel to the axes or a maximum iteration count is reached.

The phasing matrix estimation comprises the steps of:

Creating a phasing matrix, i.e. a cycle by cycle sized matrix, that represents the interaction of the cycles with each other. This matrix is created by taking three possibilities into account. a) There is phasing with probability p; b) There is pre-phasing with probability q; and c) There is no phasing or pre-phasing 1−p−q.

The phasing matrix is created with the given probabilities and it accumulates towards the last cycles.

In an embodiment of the invention, the phasing matrix is used together with the crosstalk matrix to correct the intensity values. The maximum intensity base from these corrected intensities is called as the corresponding base, i.e. A, C, G or T.

In an embodiment of the invention, a gradient based base calling is used. In some cases, a high intensity value for the wrong base due to confounding factors like spectral crosstalk is observed.

For example, in the reference spectra for a ‘C’, the 6th channel would have highest intensity while the 5th still has some intensity value due to crosstalk. And vice versa, for the ‘A’ nucleotide. However, since the sum of squared differences, SSD, method does not consider the difference direction between two channels, because of the square operation, the wrong base may be called as they would have the same error contribution.

In an embodiment of the invention, the gradient of the intensity profiles and the reference spectra are calculated, to get values that can take the difference direction between channels into consideration. Then, the similarity is calculated in the same way with the SSD method.

In some cases, a high intensity value for the wrong base is observed due to confounding factors like spectral crosstalk. For example, in the reference spectra for a ‘C’, 6th channel would have highest intensity while 5th still has some intensity value due to crosstalk. And vice versa, for the ‘A’ nucleotide. However, since the SSD method does not consider the difference direction between two channels because of the square operation, the wrong base may be called as they would have the same error contribution. To cope with this, the gradient of the intensity profiles is calculated, and the reference spectra are calculated to get values that can take the difference direction between channels into consideration. Then, the similarity is calculated in the same way with the SSD method.

### Spectral Unmixing

Spectral unmixing uses the assumption of observed intensities being a linear combination of the original nucleotide compositions. Therefore, it calculates the ratios of the different nucleotides by unmixing the spectra.

### Square Difference

Sum of squared differences of channel values of the bead intensity spectra and the reference spectra is calculated. The nucleotide with the smallest distance (i.e. highest similarity) is chosen as the base.

In an embodiment of the invention machine learning methods 500 are employed for base calling. In a preferred embodiment of the invention, a machine learning module is used in the base calling pipeline as described above to increase the number of optical barcodes matches by exploiting the already matched good barcodes from any one or more of the other methods described above.

For every experiment, optical barcodes that match the Illumina sequences with the probabilistic modelling method are taken as a machine learning dataset. The dataset is split into training, preferably 80%, and testing, preferably 20%, sets and a model is trained and tested using this split, preferably also with cross validation.

A classifier is trained for every cycle, and these classifiers can be used to predict bases for unmatched data points. For the training, neural networks are used, preferably based on random forests and/or gradient boosting trees models. Preferably, for the training one or more of: neural networks, random forests and gradient boosting trees are used as models. After the training the best performing model is used to predict the bases for every barcode in the current sample.

For the input data points, a combination of raw intensities, intensities after background correction and normalized intensities are used. Furthermore, previous cycle intensities are added as features for every consecutive cycle to include the phasing effects.

If beads produced through the combinatorial barcoding procedure described above are used for making pucks, the resulting basecalls can be matched to known barcodes used to generate beads, in order to correct errors.

### Matching of Optical Sequencing and Illumina Data

After processing the data at both sides of the assay, the barcode sequences obtained with the microscopy imaging need to be matched with those from the Illumina sequencing.

If everything would work perfectly, there would be no need to develop tools that identify the barcodes in both sides of the platform.

In embodiments of the invention, this is performed using either the machine learning approaches described in the section above, or by using methods under the framework of optimal transport.

### Machine Learning Methods

As detailed above, the machine learning module of the base calling pipeline aims to increase the number of optical barcode matches by exploiting the already matched good barcodes from other methods. For every experiment, optical barcodes that match the Illumina sequences with the probabilistic modelling method are taken as the machine learning dataset.

For the input data points, a combination of raw intensities, intensities after background correction and normalized intensities are used. Furthermore, previous cycle intensities are added as features for every consecutive cycle to include the phasing effects.

Furthermore, in an embodiment of the invention machine learning methods are employed for the matching between the two barcode sets, i.e. optical/microscopy and Illumina. Preferably, this matching is performed with various string distance metrics, e.g. the Levenshtein distance, for a fuzzy matching mechanism.

This is complemented with data from the optical side, i.e. raw & normalized intensities, bases called, etc.; and the available data in the Illumina side, i.e. bases called, “phred” scores from the “.fastq” file, gene expression; that can be combined to train an LSTM network. It has been found that the machine learning approach outperforms the optimal-transport based approach.

### Optimal Transport Methods

In computational science the optimal transport framework is used to estimate the minimum cost of a process, such as moving a pile of dirt from one place to another with the minimum effort, i.e. computational cost.

In an embodiment of the invention, the optimal transport framework is used to estimate the best matching of the two sets of barcodes between the optical decoded and the Illumina sequenced cellular barcodes.

For this, first a distance matrix is calculated for each side of the data. This distance matrix can be computed in different ways and with different features. Preferably a Hamming distance between two barcode sequences is used, but other features can be readily employed, e.g. number of diverse nucleotides, or consecutive nucleotides, combinations etc.

Each distance matrix is then a symmetric N×N matrix for N barcodes, with 0 in the diagonal and the Hamming distance between barcodes (i, j) in the i-th row and j-th column and due to symmetry also in j-th row and i-th column. The distance matrices are the input for the optimal transport framework.

The output is a list of barcode pairs from each data side, together with their associated Hamming distance.

### 3D Alignment of Consecutive Sections

The virtual representations of the consecutive 2D tissue sections are then computationally aligned to create a three-dimensional representation 700 of the tissue. The 3D representation can be used for visualization S5 and exploration of the gene expression data, but also for further downstream analyses S6, which are important for investigating the heterogeneity of tissues in space.

To achieve the 3D alignment, established methods of computer vision are used, which were first developed for alignment and registration of images. The aspect of the invention selects automatically a small number of genes of high entropy, renders them as images and performs alignment using Scale Invariant Feature Transform, which in computer vision is usually used for panorama alignment, preferably followed by a global optimizer that was developed for aligning large electron microscopy acquisitions. The aspect can be applied to an arbitrary number of sections and is independent of the number of beads captured in each section/puck, as well as of the overlapping area between the sections.

Techniques from Optimal Transport theory can also be employed for this step, as they naturally allow for image registration.

### Handling and Visualization 3D Gene Expression Data

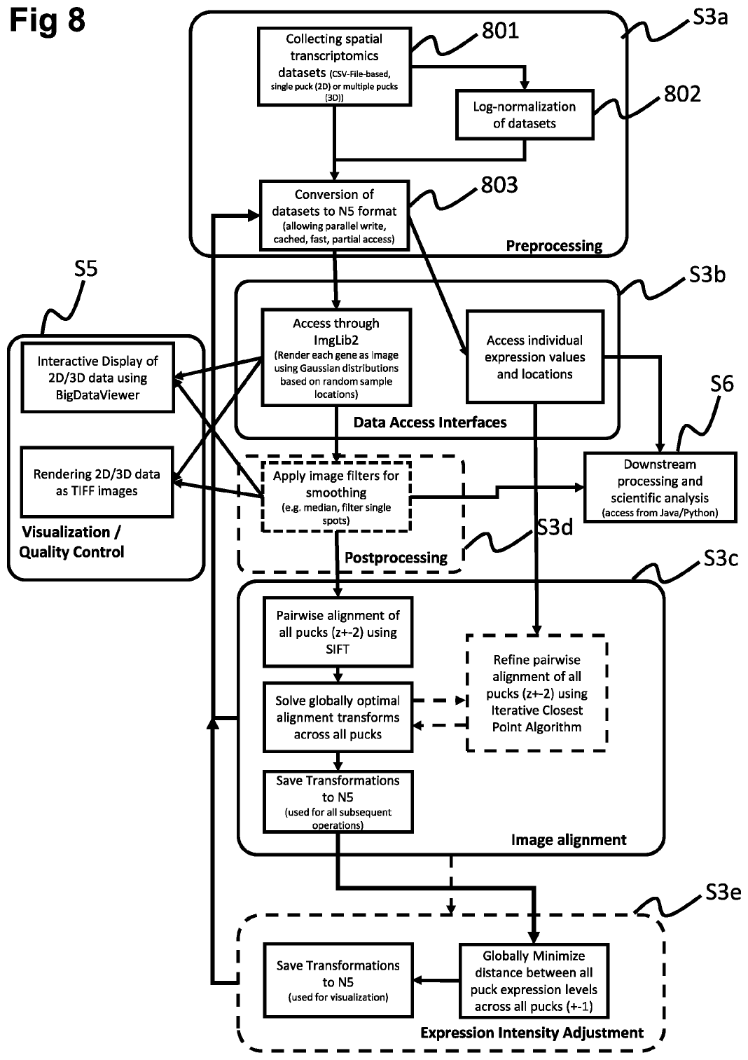

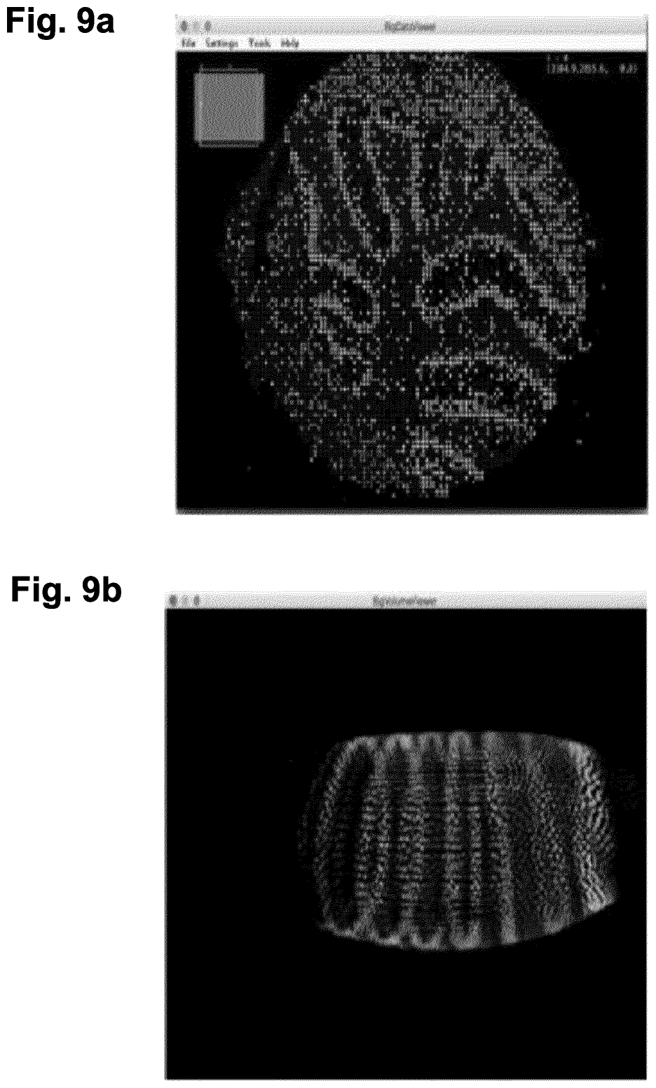

The aligned sections can be visually represented in a 3D reconstruction, see FIG. 9. FIG. 9a depicts the gene expression on a 2D section of brain cerebellum and for the gene Pcp4. Each bright spot represents a position on the puck where a bead is located and where the Pcp4 is expressed. FIG. 9b illustrates a 3D representation of a Drosophila embryo with two genes highlighted: eve and ftz. Each location that is bright represents a true location of a cell in the embryo and expresses either eve or ftz.

According to the invention, a framework based on ImgLib2, BigData Viewer and N5 is used, which allows to efficiently store, fetch, display, and run algorithms on high-dimensional spatially-resolved sequencing data. The framework can scale to the Petabyte range is therefore set up for scaling up sequencing effort. Furthermore, aforementioned framework allows seamless integration of spatially-resolved sequencing data with imaging data. Integration requires image alignment which is using existing tools for image registration that can be applied to the data using the developed framework.

### IV. Further Processing

### Data QC

In an embodiment of the invention the Quality Control, OC, is performed with standard tools, such as FastQC. However, preferably additional Quality Controls are performed at both sides of the data.

In an embodiment of the invention, for the imaging data, there are various QCs regarding the channel intensities, how they're spatially distributed on the Pucks, as well as entropic measures that measure if the recovered barcodes are meaningful or not.

In an embodiment of the invention, for the sequencing data, there are QCs based on entropic measures, nucleotide compositions for every cycle of the barcode and so on. Preferably, machine learning is employed, which uses all existing good data that has been accumulated through the years with Drop-seq, existing good and bad data from imaging experiments, as well as synthetic good and bad data to build a model that identifies if a given barcode at each side, i.e. imaging or sequencing, is prone to be erroneous or not.

### Storage of 3Denes Data

It is preferred to comply with legal issues and carefully store the data. In particular, there is a need to encrypt the data, especially the part which contains RNA or DNA sequences and that can potentially be traced back to the individual (patient or other).

### Methods for Comparing and Integrating Datasets

A deep learning method are provided for integrating and identifying patterns from single-cell omics data sets. This method learns patterns from RNA/DNA/Proteome/Metabolome that distinctively identify pathological regions. Using these methods samples or sample sections from multiple patients can easily be compared and cataloged based on their molecular profiles.

Methods for Automated Data Annotation (e.g. Cell Types, Tissue Areas)

Methods are provided for identifying cell types and tissue areas for spatial sequencing and single-cell sequencing. These methods rely on signature gene sets mined from large datasets. The numerical values of gene expressions are passed through a mathematical function that provides the likelihood of the cell being a certain type. These functions are provided for tumor-micro-environment associated cells such as immune system cells, fibroblasts, and vascular endothelial cells.

### Methods for Anonymization

Any patient metadata, e.g. gender, sex, smoking status etc., will have to be anonymized. The reason for this is that the patient metadata is used as part of our machine learning and computational methods that use sequencing and imaging data. Methods for anonymizing patient data without the loss of the statistical structure of the data are provided. The data is passed through a random transformation, the machine learning algorithms are not aware of this transformation but can perform identically. The user needs a key generated by the algorithm to apply the same transformation on new data or back-transform the data supplied to machine learning to its original structure.

### Methods for Interpretation Prediction of Metadata

The patterns such as the fraction and location of immune cell types, mutations and cancer-related abnormalities obtained from data analysis are used to predict survival and disease severity information. The numerical values of these patterns passed through a mathematical function defines the survival probability for patients.

What has been described and illustrated herein are embodiments of the invention along with some of variations. The terms, descriptions and figures used herein are set forth by way of illustration only and are not meant as limitations. Those skilled in the art will recognize that many variations are possible within the spirit and scope of the invention, which is intended to be defined by the following claims—and their equivalents—in which all terms are meant in their broadest reasonable sense unless otherwise indicated.

All references cited herein are herewith incorporated by reference in their entirety.

The present invention in particular relates to the following items:
1. A method for analyzing spatial abundance of poly-A containing RNA in a tissue sample from a subject, comprising the steps of
   (a) providing a plurality of consecutive sections, preferably cryosections, of said tissue sample,
   (b) producing a plurality of array structures by depositing for each array structure beads with an average diameter of from 1 to 100 μm on a solid support, wherein each bead comprises at least 1000 attached oligonucleotides and wherein each of the at least 1000 attached oligonucleotides of each bead comprises:
      (i) a bead identification sequence that is common to all at least 1000 oligonucleotides on each bead and that is unique to each bead in the respective array structure, and
      (ii) a poly-T sequence to capture mRNA molecules in said sample,
   (c) identifying for each array structure the bead identification sequence and associated two-dimensional position on the solid support of individual beads of the beads deposited on the solid support by performing a sequence-by-synthesis technique using a microscope,
   (d) contacting each array structure of the plurality of array structures with a section of the plurality of sections of said tissue sample and permeabilizing the tissue sections, thereby capturing poly-A containing RNA in said sample by said oligonucleotides attached to the beads,
   (e) sequencing for each array structure the RNA molecules bound to the oligonucleotides of the beads and the associated bead identification sequence for each RNA sequenced,
   (f) matching for each array structure the bead identification sequence determined in steps (c) and (e) wherein a two-dimensional position in the array structure is assigned to the nucleotide sequence of each captured RNA,
   (g) aligning the two-dimensional sequence data obtained in step (f) for consecutive sections thereby obtaining spatially-resolvable RNA abundance data from the tissue sample, wherein said aligning comprises performing a transformation on one or more reference of poly-A containing RNAs in the two-dimensional sequence data obtained in step (f) for consecutive sections.
2. The method of item 1, wherein the poly-A containing RNA is mRNA.
3. The method of items 1 to 2, wherein the beads have an average diameter of from 1 to 30 μm, preferably the beads have an average diameter of from 1 to 10 μm, more preferably 10 μm.
4. The method of items 1 to 3, wherein the solid support has a diameter of from 1 to 100 mm, preferably 1 to 40 mm, more preferably from 1 to 10 mm, even more preferably about 3 mm.
5. The method of items 1 to 4, wherein the solid support is an adhesive plastic or glass surface or Polydimethylsiloxane, PDMS, matrix.
6. The method of items 1 to 5, wherein each bead comprises between 1×10³ to 1×10⁹ attached oligonucleotides, preferably between 1×10⁵ and 1×10⁸ attached oligonucleotides, more preferably between 1×10⁷ and 1×10⁸ attached oligonucleotides, even more preferably about 3×10⁷ attached oligonucleotides, and/or wherein the oligonucleotides are DNA oligonucleotides.
7. The method of items 1 to 6, wherein the beads are polystyrene, Poly(methyl methacrylate), PMMA, or glass beads and/or, wherein the beads form a monolayer on the solid support.
8. The method of items 1 to 7, wherein each array structure comprises of from 10,000 to 10,000,000 beads, preferably 50,000 to 200,000 beads, more preferably about 100,000 beads.
9. The method of items 1 to 8, wherein sequencing of the RNA molecules in step (e) comprises reverse transcription to obtain cDNA attached to the oligonucleotides of the beads and sequencing the cDNA molecules by a next generation sequencing, NGS, technique, preferably wherein the NGS technique is Sequencing-by-Synthesis, SBS.
10. The method of items 1 to 9, wherein in step (f) an Optimal transport Problem based approach is used and/or wherein in step (g) a Scale-Invariant Feature Transform algorithm is used.
11. A method for the computer-implemented analysis of spatial abundance of poly-A containing RNA in a tissue sample, comprising the steps of:
   i) obtaining
      (i1) imaging data of a plurality of consecutive sections of said tissue sample, and
      (i2) two-dimensional sequencing data of said poly-A containing RNA in said sections, preferably two-dimensional quantitative gene expression data, preferably in a method according to any one of items 1 to 10,
   ii) registering the imaging data and detecting the two-dimensional position of beads in said imaging data, and employing a first machine learning method to obtain a first barcode set from the imaging data,
   iii) processing the two-dimensional sequencing data to obtain a second barcode set from the sequencing data,
   iv) processing the first and second barcode sets by an optimal transport framework and/or supervised machine learning to match the data sets to each other and to obtain matched barcodes,
   v) outputting based on the matched barcodes a matrix holding the expression values for each gene that was identified in each bead found in the data.
12. The method of item 11, further comprising the step of visualizing the output in a three-dimensional representation of said tissue sample.
13. A data processing system comprising means for carrying out the steps of the method of item 11.
14. A computer program product comprising instructions which, when the program is executed by a computer, cause the computer to carry out the steps of the method of item 11.
15. A computer-readable storage medium comprising instructions which, when executed by a computer, cause the computer to carry out the steps of the method of item 11.

1-16. (canceled)

17. A method for the computer-implemented analysis of spatial abundance of poly-A containing RNA in a tissue sample, comprising the steps of:
i) obtaining
(i1) imaging data of a plurality of consecutive sections of said tissue sample, and
(i2) two-dimensional sequencing data of said poly-A containing RNA in said sections,
ii) registering the imaging data and detecting the two-dimensional position of beads in said imaging data, and employing a first machine learning method to obtain a first barcode set from the imaging data,
iii) processing the two-dimensional sequencing data to obtain a second barcode set from the sequencing data,
iv) processing the first and second barcode sets by an optimal transport framework and/or supervised machine learning to match the data sets to each other and to obtain matched barcodes,
v) outputting based on the matched barcodes a matrix holding the expression values for each gene that was identified in each bead found in the data.

18. The method of claim 17, further comprising the step of visualizing the output in a three-dimensional representation of said tissue sample.

19. The method of claim 17, wherein step 1) is performed in a method comprising the steps of
(a) providing a plurality of consecutive sections of said tissue sample,
(b) producing a plurality of array structures by depositing for each array structure beads with an average diameter of from 1 to 100 μm on a solid support,
wherein each bead comprises at least 1000 attached oligonucleotides and wherein each of the at least 1000 attached oligonucleotides of each bead comprises:
(i) a bead identification sequence that is common to all at least 1000 oligonucleotides on each bead and that is unique to each bead in the respective array structure, and
(ii) a poly-T sequence to capture mRNA molecules in said sample,
(c) identifying for each array structure the bead identification sequence and associated two-dimensional position on the solid support of individual beads of the beads deposited on the solid support by performing a sequence-by-synthesis technique using a microscope,
(d) contacting each array structure of the plurality of array structures with a section of the plurality of sections of said tissue sample and permeabilizing the tissue sections, thereby capturing poly-A containing RNA in said sample by said oligonucleotides attached to the beads,
(e) sequencing for each array structure the RNA molecules bound to the oligonucleotides of the beads and the associated bead identification sequence for each RNA sequenced,
(f) matching for each array structure the bead identification sequence determined in steps (c) and (e) wherein a two-dimensional position in the array structure is assigned to the nucleotide sequence of each captured RNA,
(g) aligning the two-dimensional sequence data obtained in step (f) for consecutive sections thereby obtaining spatially-resolvable RNA abundance data from the tissue sample, wherein said aligning comprises performing a transformation on one or more reference of poly-A containing RNAs in the two-dimensional sequence data obtained in step (f) for consecutive sections.

20. A data processing system comprising means for carrying out the steps of the method of claim 17.

21. A computer program product comprising instructions which, when the program is executed by a computer, cause the computer to carry out the steps of the method of claim 17.

22. A computer-readable storage medium comprising instructions which, when executed by a computer, cause the computer to carry out the steps of the method of claim 17.

23. A method for analyzing spatial abundance of poly-A containing RNA in a tissue sample from a subject, comprising the steps of
(a) providing a plurality of consecutive sections of said tissue sample,
(b) producing a plurality of array structures by depositing for each array structure beads with an average diameter of from 1 to 100 μm on a solid support,
wherein each bead comprises at least 1000 attached oligonucleotides and wherein each of the at least 1000 attached oligonucleotides of each bead comprises:
(i) a bead identification sequence that is common to all at least 1000 oligonucleotides on each bead and that is unique to each bead in the respective array structure, and
(ii) a poly-T sequence to capture mRNA molecules in said sample,
(c) identifying for each array structure the bead identification sequence and associated two-dimensional position on the solid support of individual beads of the beads deposited on the solid support by performing a sequence-by-synthesis technique using a microscope,
(d) contacting each array structure of the plurality of array structures with a section of the plurality of sections of said tissue sample and permeabilizing the tissue sections, thereby capturing poly-A containing RNA in said sample by said oligonucleotides attached to the beads,
(e) sequencing for each array structure the RNA molecules bound to the oligonucleotides of the beads and the associated bead identification sequence for each RNA sequenced,
(f) matching for each array structure the bead identification sequence determined in steps (c) and (e) wherein a two-dimensional position in the array structure is assigned to the nucleotide sequence of each captured RNA,
(g) aligning the two-dimensional sequence data obtained in step (f) for consecutive sections thereby obtaining spatially-resolvable RNA abundance data from the tissue sample, wherein said aligning comprises performing a transformation on one or more reference of poly-A containing RNAs in the two-dimensional sequence data obtained in step (f) for consecutive sections.

24. The method of claim 23, wherein the poly-A containing RNA is mRNA.

25. The method of claim 23, wherein the beads have an average diameter of from 1 to 30 μm.

26. The method of claim 23, wherein the solid support has a diameter of from 1 to 100 mm.

27. The method of claim 23, wherein the solid support is an adhesive plastic or glass surface, or a polydimethylsiloxane (PDMS) matrix.

28. The method of claim 23, wherein each bead comprises between 1×10³ to 1×10⁹ attached oligonucleotides and/or wherein the oligonucleotides are DNA oligonucleotides.

29. The method of claim 23, wherein the beads are polystyrene, Poly(methyl methacrylate), PMMA, or glass beads and/or, wherein the beads form a monolayer on the solid support.

30. The method of claim 23, wherein each array structure comprises of from 10,000 to 10,000,000 beads.

31. The method of claim 23, wherein sequencing of the RNA molecules in step (e) comprises reverse transcription to obtain cDNA attached to the oligonucleotides of the beads and sequencing the cDNA molecules by a next generation sequencing (NGS) technique or a sequencing-by-synthesis (SBS) technique.

32. The method of claim 23, wherein in step (f) an Optimal transport Problem based approach is used and/or wherein in step (g) a Scale-Invariant Feature Transform algorithm is used.
